(19) United States
(12) Patent Application Publication
(10) Pub. No.: US 2019/0125256 A1
(43) Pub. Date: May 2, 2019

(54) Automatic Drug Administration Device and Method

(71) Applicant: Hays, Inc., Onsted, MI (US)

(72) Inventor: Gregory Hays, Onsted, MI (US)

(21) Appl. No.: 16/173,273

(22) Filed: Oct. 29, 2018

Related U.S. Application Data
(60) Provisional application No. 62/578,689, filed on Oct. 30, 2017.

(51) Int. Cl. A61B 5/00; A61B 5/0205; A61M 11/00; A61B 5/08; A61B 5/11; A61B 5/1455; G16H 20/10

(52) U.S. Cl. CPC A61B 5/4839; A61B 5/0205; A61M 11/00; A61B 5/08; G16H 50/30; A61B 5/14551; G16H 20/10; A61B 5/746; A61B 5/11

(57) ABSTRACT

A device for detecting an overdose and automatically administering a drug includes a sensor configured to measure a condition of a user indicative of overdose. The device includes a container that retains the drug and a drug delivery device fluidly connected to the sensor and the container. The device includes a pressurized gas container retaining pressurized gas. The pressurized gas container is configured to release the pressurized gas to expel the drug from the container into the drug delivery device. The device includes a controller electrically coupled to the sensor and the pressurized gas container. The controller is configured to determine whether to release the pressurized gas. The device is wearable by the user and is portable such that movement and location of the user is not restricted

### CROSS-REFERENCE TO RELATED APPLICATIONS

This application claims the benefit of U.S. Provisional Application No. 62/578,689, filed on Oct. 30, 2017, the content of which is hereby incorporated by reference in its entirety for all purposes.

### TECHNICAL FIELD

This disclosure generally relates to the field of automatic drug administration devices and methods. The disclosure has a non-exclusive use with pharmaceutical compounds to treat narcotic overdose.

### BACKGROUND

The use of prescription opioid drugs has been on the rise since the mid-2000s resulting in the current opioid epidemic. Nearly half of all opioid related deaths have been attributed to prescriptions. Drug overdoses have since become the leading cause of death of Americans under 50, with two-thirds of those deaths caused by opioids.

A popular treatment for opioid addiction is naloxone (NARCAN®) that is primarily used as an emergency treatment for acute opioid overdose. Naloxone must be administered within minutes of the overdose to prevent brain damage or death.

### SUMMARY

One aspect of the disclosure is a device for detecting an overdose and automatically administering a drug. The device includes a sensor configured to measure a condition of a user indicative of overdose. The device includes a container that retains the drug and a drug delivery device fluidly connected to the sensor and the container. The device includes a pressurized gas container retaining pressurized gas, the pressurized gas container being configured to release the pressurized gas to expel the drug from the container into the drug delivery device. The device includes a controller electrically coupled to the sensor and the pressurized gas container, wherein the controller is configured to release the pressurized gas upon a predetermined event.

Another aspect of the disclosure is a device for detecting an overdose and automatically administering a drug. The device includes a sensor configured to monitor breathing of a user and a container retaining the drug. The device includes a nasal cannula fluidly connected to the container and the sensor. The device includes a drug supply device configured to carry the drug through the nasal cannula and to the user. The device includes a mobile alert device and a controller electrically coupled to the mobile alert device, the sensor, and the drug supply device. The controller is configured to the drug supply device to supply the drug to the user through the nasal cannula upon a predetermined event, such as undetectable respiration. The controller is configured to use the mobile alert device to send a notification to an external device in response to supplying the drug to the user.

Another aspect of the disclosure is a method for detecting an overdose and automatically administering a drug. The method includes measuring a condition of a user indicative of an overdose using a sensor. The method includes receiving, at a controller, data indicative of the condition of the user from the sensor. The method includes in response to the controller determining that the condition of the user meets a threshold, dispensing the drug to the user using a drug supply device and a drug delivery device.

Another method for detecting an overdose and automatically administering a drug comprises providing to a user a portable device including a nasal cannula connected to a sensor and a drug delivery means to be worn by the user during opioid use; monitoring using the sensor respiration of the user through the nasal cannula; when the sensor detects a respiration event, automatically activating the drug delivery means to propel a drug to the user through the nasal cannula to reestablish respiration.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a diagram of a device configured to detect an overdose and automatically administer a drug according to one embodiment.

FIG. 2 is a block diagram of an example of communication between the device and one or more external devices.

FIG. 3 is a diagram of the device connected to the user according to one embodiment.

FIG. 4 is a diagram the device according to one embodiment where the detection means includes an RIP band.

FIG. 5 is a diagram of a device configured to detect an overdose and automatically administer a drug according to another embodiment.

FIG. 6 is a flowchart of a process for detecting an overdose and automatically administering a drug.

### DETAILED DESCRIPTION

When a drug user overdoses, medical assistance often arrives too late to prevent brain damage or death of the drug user. Accordingly, there is a need for a portable device that can be worn or carried by the drug user that detects a potential overdose and automatically administers an antidote, such as naloxone, without the intervention of the drug user, to save the life of the drug user during an acute opioid overdose. Devices disclosed herein are configured to be portable and worn by the drug user, to monitor the user for conditions indicative of drug overdose, to determine whether an indicator of drug overdose exists, and to deliver a drug such as naloxone to the user in response to determining that the indicator exists. In some embodiments, the devices may send a notification and the user's location to one or more external devices.

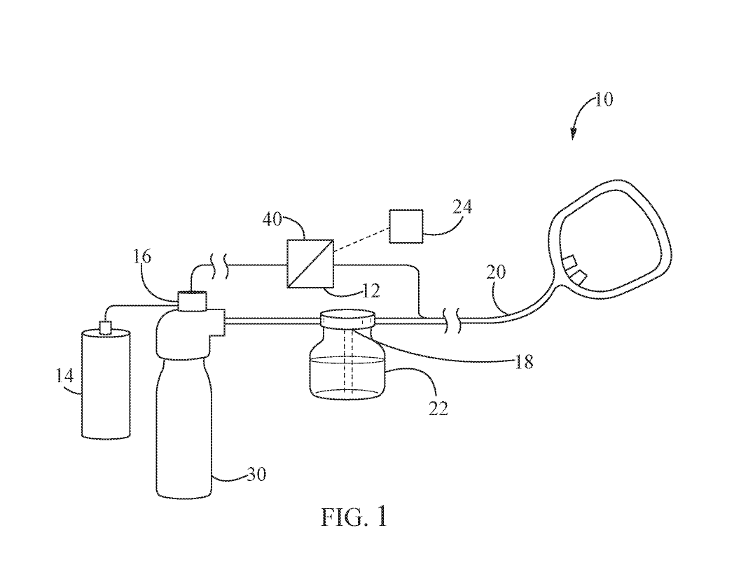

FIG. 1 is a diagram of a device 10 configured to detect an overdose and automatically administer a drug without human intervention according to one embodiment. The device 10 is meant to be portable and is worn or carried by a user who is taking harmful substances with a potential for overdosing, such as opioid or heroin. In some embodiments, the device 10 can be connected to the user. For example, the device 10 can be connected to the user around the user's waist using a belt clip (not shown), around the user's chest using an elastic band, or can be attached to any other portion of the user in any other suitable manner. In some embodiments, the device 10 can be carried by the user. For example, the device 10 can be carried in a pocket of a shirt, jacket, pants, or any other article of clothing. The device 10 can also be disposed in a housing, bag, purse, fanny pack, or any other container.

The device 10 includes a detection means 40 for measuring one or more conditions of the user. For example, the detection means 40 may measure conditions indicative of user overdose (e.g. a hypoxic state or an imminent hypoxic state). The detection means 40 can include a respiration sensor, a carbon dioxide (CO₂) sensor, a Respiratory Inductance Plethysmography (RIP) band, a pulse oximeter, one or more other sensors, or a combination thereof. In the illustrated example, the detection means 40 is a CO₂ sensor.

The device 10 also includes a drug delivery device 20. The drug delivery device 20 facilitates delivery of the drug from the device 10 to the user. The drug delivery device 20 can include any nasal cannula, breathing mask, or any other suitable medical device allowing the user to inhale the drug into the user's respiratory system. In the illustrated example, the drug delivery device 20 includes a high-flow nasal cannula having one end connected to the device 10 and an opposing end portion configured to be retained in the nostrils of the user. In some embodiments, the detection means 40 may be fluidly connected to the drug delivery device 20. For example, the drug delivery device 20 may include a nasal cannula and the detection means 40 may include a respiration sensor disposed inside the nasal cannula. In some embodiments, the detection means 40 may not be fluidly connected with the drug delivery device 20.

In some embodiments, the drug delivery device 20 may include a needle, syringe, intravenous line (IV) or any other injection device that injects the drug into the user. A container retaining the drug can be positioned on or near the user's body at a location in which, when activated, the injection device would penetrate the user's skin and inject the drug into the user.

The device 10 includes a container 22 fluidly connected with the drug delivery device 20 such that the drug delivery device 20 can facilitate transportation of the drug from the container 22 to the user. The container 22 retains (e.g. holds) the drug. The drug can include any medication, antidote, or any drug including but not limited to naloxone.

The device 10 includes a drug supply device 30 for supplying the drug to the user. For example, the drug supply device 30 may transport the drug from the container 22, through the drug delivery device 20, and to the user. In one embodiment, the drug supply device 30 is configured to aerosolize or atomize the drug. The drug supply device 30 can include, for example, a pressurized gas container, a plunger, a fan, any other suitable device, or combination thereof.

In FIG. 1, the drug supply device 30 is illustrated as being a pressurized gas container. The pressurized gas container retains pressurized gas including air, oxygen, CO₂, or any other gas. In some embodiments, the pressurized gas container is configured to release the pressurized gas to expel the drug from the container 22, through the drug delivery device 20, and to the user. In some embodiments, the drug supply device 30 may include a pressure sensor adapted to measure the pressure of the pressurized gas inside the pressurized gas container. The pressure sensor can be used to measure remaining doses of the drug, or to confirm that there is adequate gas left in the pressurized gas container to deliver the drug to the user if needed. In some embodiments, the device 10 may not include the drug supply device 30 and the drug may be retained in a pressurized container that is configured to deliver the drug to the user.

In some embodiments, the controller 12 may activate an electronically activated valve 16 fluidly connected to the drug supply device 30 to supply the drug to the user. For example, in response to engaging the electronically activated valve 16, the pressurized gas inside the pressurized gas container may be released into the drug delivery device 20 to deliver the drug to the user. In some embodiments, the container 22 of the drug may include a venturi valve 18 to assist in the administration of the drug by increasing its velocity while exiting the container 22.

Once the device 10 is connected to the user, the device 10 is intended to be in continuous use. That is, once connected to the user, the device 10 can, as further described below, monitor the user and deliver the drug to the user in response to detection of an adverse medical condition, such as overdose. In some embodiments, the drug delivery device 20 (e.g. the nasal cannula or the breathing mask) is the only part of the device 10 that is viewable by others.

In some embodiments, the device 10 can be set up on a user who may not be able to physically and/or consciously engage with (e.g. set up, wear, turn on and off, etc.) the device 10. In other words, the drug delivery device 20 can be placed on the user by a third party such that the third party does not have to monitor the user to prevent the user from overdosing. For example, the third party may place the nasal cannula in the user's nostrils, place the breathing mask over the user's nose and/or mouth, or place the injection device in or near the user. In some embodiments, the user can himself or herself place the drug delivery device 20 in an operable position prior to and/or during the use of harmful substances so the device 10 can be used to prevent potential overdoses.

The device 10 includes a controller 12 electrically coupled to any described component including but not limited to the detection means 40, the drug supply device 30, the drug delivery device 20, and a power source 14. The power source 14 may include one or more batteries of any type or any other suitable power source. In some embodiments, the power source 14 may include multiple power sources, each powering one or more described components.

In some embodiments, the device 10 can include one or more indicators configured to communicate information to the user and/or third parties. For example, the one or more indicators may indicate the occurrence of an overdose, battery life remaining in the power source 14, an amount of drug remaining in the device 10, the detected CO₂ level of the breach of the user, and/or the respiration rate of the user. The indicator can be powered by the power source 14 and can be electrically coupled to the controller 12. The indicator can include, for example, a digital screen display, a speaker, or lights. In some embodiments, the indicator can automatically generate a notification in response to the controller 12 determining that the user has overdosed based on signals sent by the detection means 40. For example, the lights may flash or the display may generate a notification. In some embodiments, as further described below, the device 10 may be capable of networked communication. As such, notifications can be sent to one or more external devices such as a cloud-based server, a mobile device, an email system, other networked devices or systems, or a combination thereof.

In some embodiments, the controller 12 may include a timer configured to track the amount of time that elapses since the drug was last administered to the user (e.g. dispensed). The time may be displayed on the indicator, for example to assist a medical professional treating the user. In some embodiments, the device 10 may administer the drug to the user based on the time elapsed since the drug was last administered. For example, the device 10 can administer the drug to the user in response to the detection means 40 measuring a condition of the user indicative of overdose. The detection means 40 can continue to monitor the condition of the user after administration of the drug. Based on a determination by the controller 12 that the user's condition is not improving (e.g. the user's respiration rate and/or CO₂ level do not meet respective thresholds) and based on the expiration of a predetermined time period since the previous administration of the drug, the device can administer the drug again to the user.

In some embodiments, the device 10 may prevent administration of the drug to the user based on the time elapsed since the drug was last administered. For example, if a minimum time period has not elapsed since the last administration of the drug, the controller 12 may prevent administration of the drug to the user even if the detection means 40 measures a condition of the user indicative of overdose (e.g. the user's respiration rate and/or CO₂ level do not meet respective thresholds). The indicator may produce a notification indicative of the user's condition if the controller 12 determines that the measured condition indicates an overdose and the device 10 does not administer the drug.

The detection means 40 can include a respiration sensor that is configured to monitor the respiration rate of the user. The respiration rate sensor can transmit a signal indicative of the respiration rate to the controller 12. The detection means 40 can include a CO₂ sensor that is configured to monitor the CO₂ level in the user's breath and to transmit a signal indicative of the CO₂ level to the controller 12. The detection means 40 can include the RIP band configured to measure the movement of the chest and abdominal wall and to transmit a signal indicative of the movement to the controller 12. The detection means 40 can include a pulse oximeter to measure oxygen level and heart rate and to transmit a signal indicative of the oxygen level and heart rate to the controller 12. The controller 12 is configured to determine if the user has entered, or is about to enter, a hypoxic state based on at least one of the measured conditions indicative of overdose. The measured conditions may include CO₂ level, chest movement, abdominal movement, respiration rate, other conditions, or any combination thereof. For example, if the controller 12 determines that the CO₂ level is above a first threshold and/or determines that the respiration rate, oxygen level, chest movement, and/or abdominal movement is below a second threshold, the controller 12 transmits a signal to the drug supply device 30 to supply the drug to the user through the drug delivery device 20.

The device 10 can include a mobile alert device 24. The device 10 can also include a GPS device configured to determine a location of the device 10. The GPS device may be included in the mobile alert device 24. The mobile alert device 24 sends a notification (including, for example, an alert message, information indicative of the user's condition measured by the detection means 40, and/or the user's location) to one or more external devices 50 or to emergency services. For example, the mobile alert device 24 can send the notification to a mobile phone in response to the device 10 determining that the user's respiratory rate is lower than a threshold. The mobile alert device 24 may include software programmed into a processor, integrated circuit, or may be otherwise executed by the device 10. In some embodiments, the mobile alert device 24 is included in the controller 12. The mobile alert device 24 can be integrated into the device 10 such that the mobile alert device 24 is included in the same housing, carrier, or any other container as the device 10. In some embodiments, the mobile alert device 24 can be included in hardware separate from the device 10 that is in communication with the device 10. For example, the mobile alert device 24 can be worn separately from the device 10, such as on a lanyard around the user's neck or carried in a pocket.

In some embodiments, the mobile alert device 24 can be activated using a command generated by the user. In some embodiments, the mobile alert device 24 can be in electronic communication with the controller 12 such that the controller 12 activates the mobile alert device 24. For example, the controller 12 can activate the mobile alert device 24 when the drug has been delivered to the user and/or when the detection means 40 measures a condition of the user indicative of an overdose. In some embodiments, the mobile alert device 24 can periodically or constantly update one or more external devices 50 with notifications.

In some embodiments, the GPS device may be enabled or disabled by an administrator having access to the GPS device. The administrator can include the user and/or a designated person other than the user. Access to the GPS device (e.g. the ability to turn the GPS device on and off) by the administrator may be password protected or otherwise secured.

The controller 12 may be in communication (e.g., using a network interface of the controller 12) with one or more of the external devices 50 including but not limited to a mobile phone, a tablet computer, a laptop computer, a notebook computer, a desktop computer, or a server computer, a cloud-based server, other networked devices or systems, or a combination thereof. In some embodiments, the user or the administrator may define specified recipients or specified external devices to which notifications may be sent. The communication may be wired or wireless (e.g., WiFi, Bluetooth, USB, HDMI, Wireless USB, Near Field Communication (NFC), Ethernet, a radio frequency, and/or other interfaces for communicating data between one or more external devices).

In some embodiments, the mobile alert device 24 will send the notification through a cellular network, for example, over 3G, 4G, 5G, CDMA, or GSM. The mobile alert device 24 can use the cellular network to send the notification to emergency services or an emergency contact. For example, the mobile alert device 24 can use the cellular network to send the notification to a monitoring center which will in turn send the notification to emergency services.

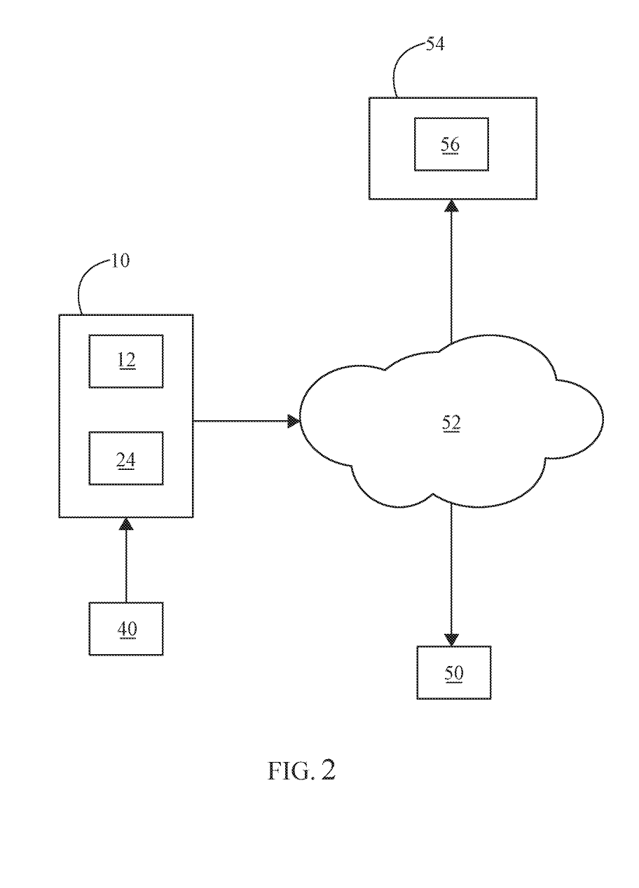

FIG. 2 is a block diagram of an example of communication between the device 10 and one or more external devices 50. In some embodiments, the controller 12 may collect data for transfer and/or storage. The data may include but is not limited to occurrence of an overdose, the user's respiration rate, the user's CO₂ level, the user's chest movement, the user's abdominal movement, time the device 10 has been in use, medication dosage delivered to the user, time since the last dosage was delivered to the user, the user's location, or any other user information described herein. The data may be transmitted to a server used to implement a software as a service product at which records of the user's data are stored. In the illustrated example, the detection means 40 transmits the data to the controller 12 and/or the mobile alert device 24 of the device 10. The device 10 transmits the data to a network 52. The data is transmitted from the network 52 to a server 54 optionally including a database 56 and/or one or more of the external devices 50. The one or more external devices 50 may include a server. The server 54 may include a hardware component or a software component (e.g. a web server).

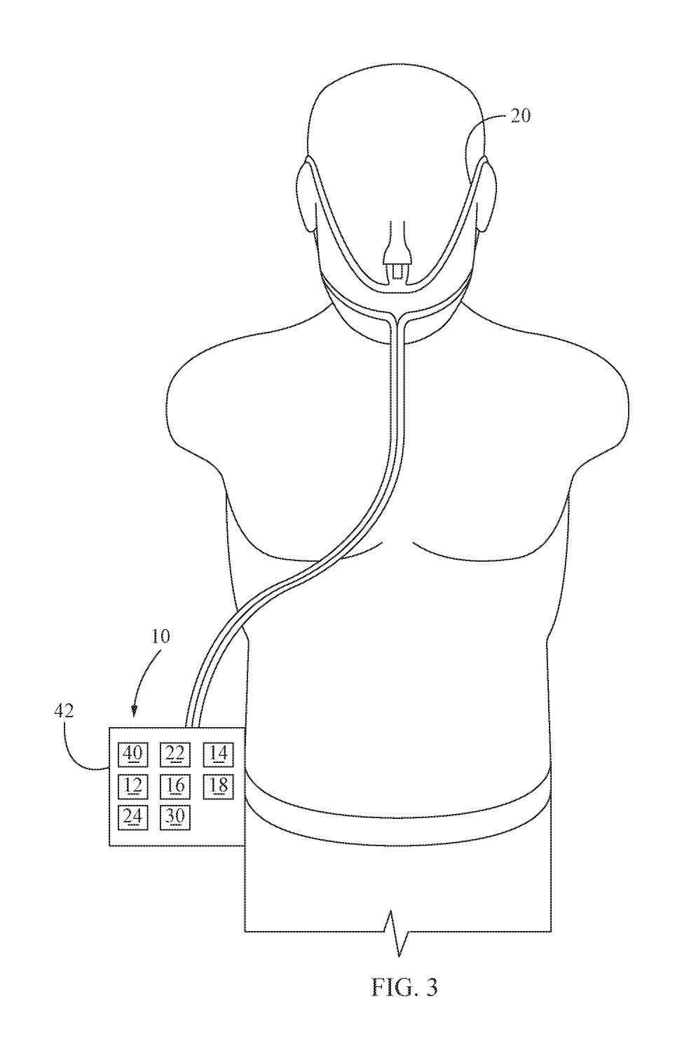

FIG. 3 is a diagram of the device 10 connected to the user according to one embodiment. Any of the described components of the device 10, including but not limited to, the controller 12, the detection means 40, the drug delivery device 20, the mobile alert device 24, the container 22, the venturi valve 18, the activated valve 16, the drug supply device 30, and the power source 14 may be disposed inside a housing 42. The housing 42 can include a bag, box, carrier, or any other container. The housing 42 can be connected to the user's belt, belt clip, belt loop, or any other article of clothing. In some embodiments, the housing 42 can be connected to the user using any fastener.

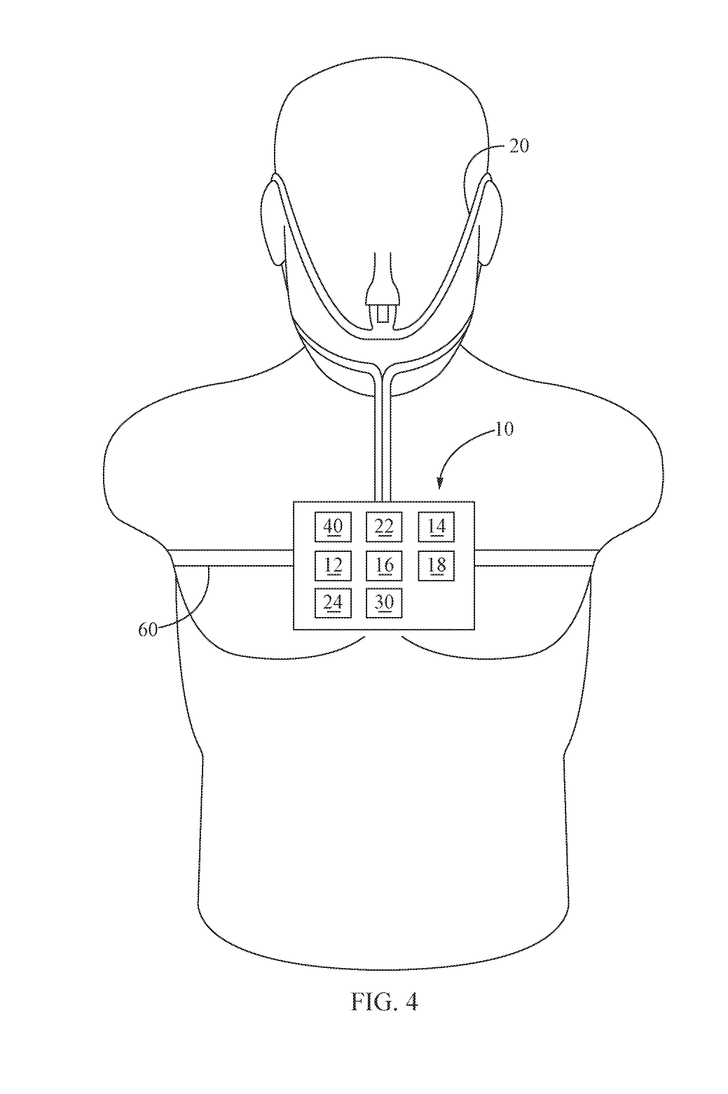

FIG. 4 is a diagram the device 10 according to one embodiment where the detection means 40 includes an RIP band 60. The RIP band 60 may include an adjustable band (e.g. a band having elastic portions or hook and loop fasteners) worn around the user's torso. In some embodiments, any other described detection means 40 can also be included, such as the CO₂ sensor. The RIP band 60 is in communication with the controller 12. Any of the described components of the device 10, including but not limited to the controller 12, the detection means 40, the drug delivery device 20, the mobile alert device 24, the container 22, the venturi valve 18, the activated valve 16, the drug supply device 30, and the power source 14 may be connected to the RIP band 60. In some embodiments, the components of the device 10 may be connected to a housing, carrier, or any other container connected to the RIP band 60.

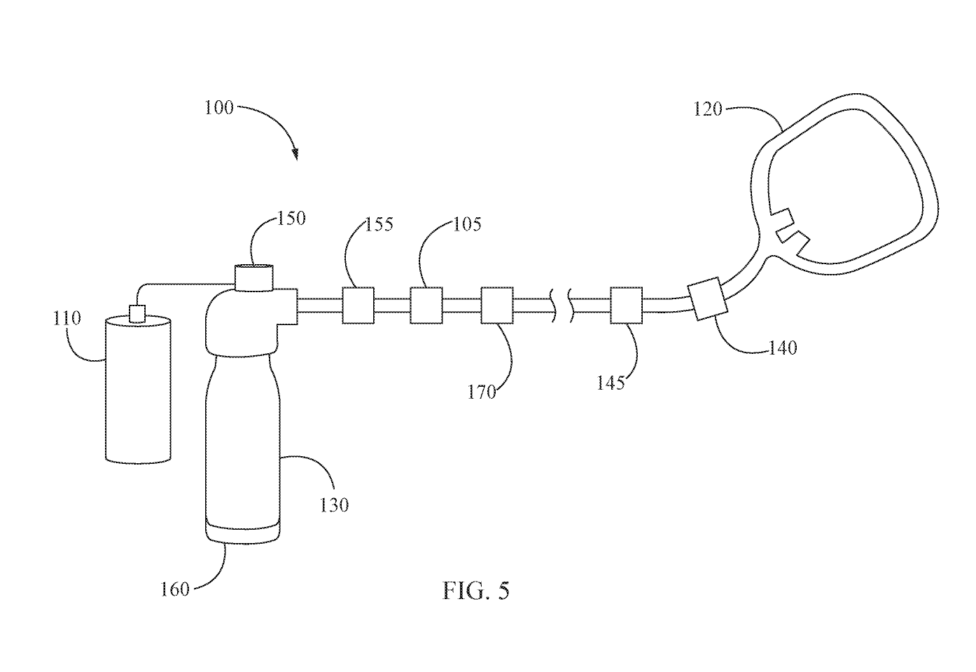

FIG. 5 is a diagram of a device 100 configured to detect an overdose and automatically administer a drug according to another embodiment. The device 100 may include features similar to those of device 10 except as otherwise described. The device 100 is a portable device that can be attached to a user, for example, by way of a belt clip (not shown) or in a pocket of a shirt, jacket or pants. Referring to FIG. 5, the device 100 includes a controller 105 and a power source 110. The device 100 also includes a nasal cannula 120 that is fluidly connected to a pressurized canister 130 that contains an antidote, for example, naloxone. The pressurized canister 130 is configured to aerosolize the drug and may be removable and/or refillable.

The nasal cannula 120 is fluidly connected to a respiration sensor 140, a carbon dioxide (CO₂) sensor 145, a pressure sensor 150, and an optional fan 155. The respiration sensor 140 and the CO₂ sensor 145 may be combined into a single sensor. The device may include only the respiration sensor 140 or only the CO₂ sensor 145, or both sensors. The pressure sensor 150 is configured to measure the pressure of the pressurized canister 130 to estimate the volume of its contents. The pressure sensor 150 may also act as a dose counter.

The controller 105 is electrically coupled to the respiration sensor 140, CO₂ sensor 145, pressure sensor 150, optional fan 155, plunger 160, and one or more indicator lights 170. The respiration sensor 140 is configured to monitor the respiration rate of the user and to transmit a signal indicative of the respiration rate to the controller 105. The CO₂ sensor 145 is configured to monitor the CO₂ level in the user's breath and to transmit a signal indicative of the CO₂ level to the controller 105. The controller 105 is configured to determine if the user has entered, or is about to enter, a hypoxic state based on the CO₂ level and/or respiration rate. If the controller 105 determines that the CO₂ level is above a first threshold and/or determines that the respiration rate is below a second threshold, the controller signals the plunger 160 to deploy.

The deployment of the plunger 160 causes a predetermined dose of aerosolized antidote to be expelled from the pressurized canister 130 into the nasal cannula 120. The antidote travels through the nasal cannula and into the nasal cavity of the user where the antidote is administered. In one example, the controller may activate the optional fan 155 to aid in drug delivery and ensure that the full dose is administered.

The pressure sensor 150 monitors the pressure of the pressurized canister 130 and/or monitors the number of doses administered. The pressure sensor 150 signals the pressure and/or number of doses administered to the controller 105. When the controller 105 determines that the pressure falls below a threshold or that a maximum number of doses have been administered, the controller 105 signals one or more indicator lights 170 to illuminate, alerting that the antidote is low or depleted. The indicator lights 170 are also configured to receive a signal from the power source 110 to indicate a low power level.

The device (i.e. device 10 or device 100) monitors the CO₂ level in the nasal cannula and/or the respiration rate of the user and/or the respiration movement of the user with the detection means 40. The device determines whether a threshold parameter relating to the detected parameter is above or below a predetermined threshold. If, for example, the CO₂ level is above a threshold, or whether the respiration rate of the user is below a threshold, the device dispenses the aerosolized antidote. If not, the device will continue to monitor the user. In addition, upon dispensing the aerosolized antidote, the device can continue to monitor the user through the nasal cannula to determine if another dose of antidote is needed.

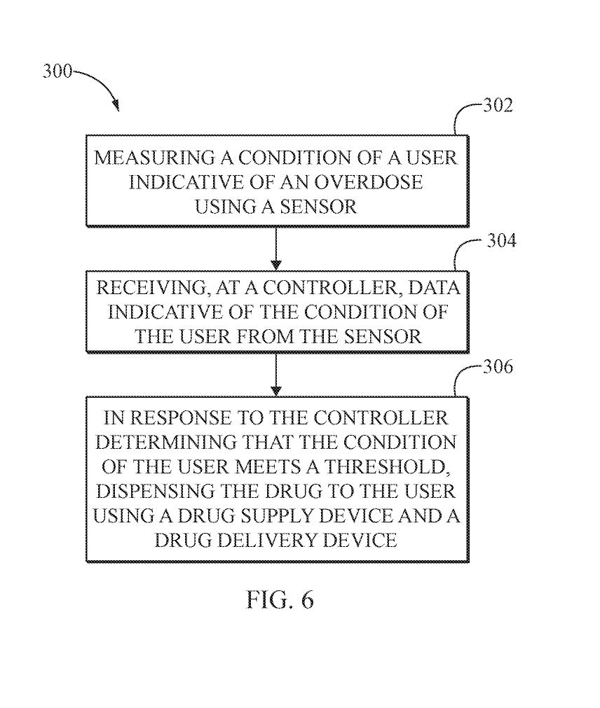

FIG. 6 is a flowchart of a process 300 for detecting an overdose and automatically administering a drug without the need for human intervention or assistance. Because the device is portable, the process 300 is performed wherever the drug user is located, such as outdoors, in a house, in a park, in an abandoned building, in his or her own bedroom or bathroom, or in a medical or treatment setting where that person cannot be monitored by a person at all times. The process 300 can be used in or by any of the devices described herein (e.g. device 10 or device 100) is described. Operation 302 of the process 300 includes measuring a condition of the user indicative of an overdose. In some embodiments, measuring the condition of the user can include measuring the CO₂ level of the user's breath, measuring the respiration rate of the user, and/or measuring the movement of the user's chest/and or abdominal wall. The respiration of the user may be measured by the detection means 40.

The operation 304 of the process 300 includes receiving data indicative of the condition of the user. Receiving the data indicative of the condition of the user may include receiving data indicative of the user's CO₂ level, respiration rate, chest movement, and/or abdominal wall movement. In some embodiments, the controller 12 may receive the data indicative of the condition of the user.

Operation 306 of the process 300 includes dispensing the drug to the user in response to determining that the condition of the user does not meet a threshold. In some embodiments, determining that the condition of the user does not meet a threshold includes determining that the CO₂ level of the breath of the user is above a first threshold and/or that the respiration rate is below a second threshold. The controller 12 may determine that the condition of the user does not meet the threshold. The device may dispense the drug to the user using the drug supply device 30 and the drug delivery device 20. If the controller 12 determines that the condition of the user does not meet the threshold, the process 300 may return to operation 302.

In some embodiments, the process 300 may further include sending a notification to one or more of the external devices 50 in response to determining that the condition of the user does not meet the threshold. The mobile alert device 24 and/or the controller 12 may send the notification.

Implementations of the controller 12 and/or the mobile alert device 24 (and the algorithms, methods, instructions, etc., stored thereon and/or executed thereby) can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors or any other suitable circuit. In the claims, the term “controller” should be understood as encompassing any of the foregoing hardware, either singly or in combination. The terms “signal” and “data” are used interchangeably. Further, portions of the controller 12 and the mobile alert device 24 do not necessarily have to be implemented in the same manner.

Further, in one aspect, for example, the controller 12 and/or the mobile alert device 24 can be implemented using a general-purpose computer or general-purpose processor with a computer program that, when executed, carries out any of the respective methods, algorithms and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

While the invention has been described in connection with certain embodiments, it is to be understood that the invention is not to be limited to the disclosed embodiments but, on the contrary, is intended to cover various modifications, combinations, and equivalent arrangements included within the scope of the appended claims, which scope is to be accorded the broadest interpretation so as to encompass all such modifications and equivalent structures as is permitted under the law. One or more elements of the embodiments disclosed may be combined with one or more elements of any other embodiment disclosed.

What is claimed is:

1. A device for detecting an overdose and automatically administering a drug, the device comprising:
a sensor configured to measure a condition of a user indicative of overdose;
a container that retains the drug;
a drug delivery device fluidly connected to the container;
a pressurized gas container retaining pressurized gas, the pressurized gas container being configured to release the pressurized gas to expel the drug from the container into the drug delivery device; and
a controller electrically coupled to the sensor and the pressurized gas container, wherein the controller is configured to activate release of the pressurized gas based on a signal from the sensor, wherein the device is wearable by the user and is portable such that movement and location of the user is not restricted.

2. The device of claim 1, wherein:
the sensor includes a CO₂ sensor configured to measure a CO₂ level in a breath of the user;
the condition includes the CO₂ level; and
the controller is further configured to release the pressurized gas in response to a determination that the CO₂ level is above a predetermined threshold.

3. The device of claim 1, wherein:
the sensor includes a respiration sensor configured to measure a respiration rate of the user;
the condition includes the respiration rate; and
the controller is further configured to expel the drug from the container into the drug delivery device in response to a determination that the respiration rate is below a predetermined threshold.

4. The device of claim 1, wherein the drug delivery device includes a nasal cannula.

5. The device of claim 1, wherein the drug delivery device includes a breathing mask.

6. The device of claim 1, wherein the pressurized gas is configured to aerosolize or atomize the drug.

7. The device of claim 1, further comprising a pressure sensor configured to measure a pressure of the pressurized gas.

8. The device of claim 7, wherein the pressure sensor is further configured to measure remaining doses of the drug.

9. The device of claim 1, wherein the sensor is one or more of a respiration rate sensor, a CO₂ sensor, and an RIP band.

10. The device of claim 1 further comprising:
a mobile alert device including a GPS device, wherein the mobile alert device sends a notification to an external device in response to the controller determining to release the pressurized gas.

11. The device of claim 10, wherein the mobile alert device sends a notification to an external device in response to a command generated by the user.

12. A device for detecting an overdose and automatically administering a drug, the device comprising:
a nasal cannula worn by a user;
a sensor configured to monitor breathing of the user using gas exhaled by the user to the nasal cannula;
a container retaining the drug;
a drug supply device configured to carry the drug through the nasal cannula and to the user;
a mobile alert device; and
a controller electrically coupled to the mobile alert device, the sensor, and the drug supply device, the controller configured to activate the drug supply device to supply the drug to the user through the nasal cannula based on output of the sensor, and configured to use the mobile alert device to send a notification to an external device in response to supplying the drug to the user, wherein
the device is wearable by the user and is portable such that movement and location of the user is not restricted.

13. The device of claim 12, wherein the sensor is one or both of a respiration rate sensor and a CO₂ sensor.

14. The device of claim 12, where in the drug supply device is a pressurized gas container holding a pressurized gas.

15. The device of claim 14, wherein the pressurized gas container includes an electronically activated valve that is actuated using a battery in response to receiving a signal from the controller.

16. The device of claim 12, further comprising:
a housing retaining the device, wherein the nasal cannula extends through at least a portion of the housing, and wherein the housing is portable with the user.

17. The device of claim 12, wherein the mobile alert device includes a GPS device and the notification sent by the mobile alert device includes a location of the device.

18. A method for detecting an overdose and automatically administering a drug, comprising:
providing to a user a portable device including a nasal cannula connected to a sensor and a drug delivery means to be worn by the user during opioid use;
monitoring using the sensor respiration of the user through the nasal cannula;
when the sensor detects a respiration event, automatically activating the drug delivery means to propel a drug to the user through the nasal cannula to reestablish respiration.

19. The method of claim 18, wherein the sensor monitors one of respiration rate or carbon dioxide level.

20. The method of claim 18, further comprising sending a notification using a mobile alert device to an external device in response to drug delivery means being activated.
